(19) United States
(12) Patent Application Publication
(10) Pub. No.: US 2004/0142910 A1
(43) Pub. Date: Jul. 22, 2004

(54) Sulfonated styrene copolymers for medical uses

(75) Inventors: David J. Vachon, Granada Hills, CA (US); Gary Wnek, Midlothian, VA (US)

Correspondence Address: HESLIN ROTHENBERG FARLEY & MESITI PC, 5 COLUMBIA CIRCLE, ALBANY, NY, 12203, US

(73) Assignee: Aegis Biosciences LLC, Palm Harbor, FL (US)

(21) Appl. No.: 10/691,117

(22) Filed: Oct. 21, 2003

Related U.S. Application Data
(60) Provisional application No. 60/420,049, filed on Oct. 21, 2002.

(51) Int. Cl. A61K 31/65; A61L 15/00; A61K 9/14

(52) U.S. Cl. 514/152; 424/486; 424/445

(57) ABSTRACT

Sulfonated styrene copolymers are useful for inhibiting elastase and/or collagenase and for promoting angiogenesis in a wound, and for controlling biological organisms on a porous surface. Compositions for these uses may include a tetracycline, an amino acid and/or a sulfonated styrene copolymer in salt form, especially an ammonium salt.

### RELATED APPLICATION

This application is a non-provisional of Provisional Patent Application Serial No. 60/420,049, filed Oct. 21, 2002, the entire contents of which are incorporated herein by reference.

### FIELD OF THE INVENTION

The invention relates to hydrophilic sulfonated styrene polymers and their use in drug delivery devices, such as moist (hydrogel) wound dressings, cavity inserts for vaginal, rectal drug delivery, oral drug delivery, drug delivery from surgically created spaces, and coatings for implantable medical devices.

### BACKGROUND OF THE INVENTION

Research has established that healing of wounds such as burns, skin ulcers, pressure sores and traumatic injuries is facilitated when the wound bed is kept moist and clean. Moist wound dressings are particularly useful for this purpose and have become an accepted therapy for treating wounds. In this context, moist means that the dressing keeps the wound moist, and not necessarily that the dressing is moist when applied to the wound. It is postulated that these dressings promote optimum physiological conditions for healing in the wound by maintaining or promoting tissue hydration. When applied to dry wounds, the dressings rehydrate desiccated tissue, either by preventing loss of water vapor from the site or by directly transferring moisture to the tissue. When applied to exuding wounds, the dressings absorb the exudate and promote hydration of tissue. Autolytic debridement of necrotic tissue and/or formation of new tissue occur more readily under these conditions. In addition, a variety of growth factors that promote wound healing are present in the exudates from the wounds (see Howell, J. M., Current and Future Trends in Wound Healing, Emerg. Med. Clin. North Amer., 10, 655-663 (1992)), and it is believed that moist wound dressings that can absorb fluids from the exudate promote healing by minimizing loss of these growth factors from the wound bed.

Several types of moist wound dressings are commercially available, including hydrogels, hydrocolloids, semipermeable adhesive films, perforated films, alginates, polysaccharide beads, and polyurethane foams. These dressing types are distinguished by physical form, mechanisms of action, and by their chemical compositions.

Hydrogel dressings are composed of water insoluble polymers having hydrophilic sites that interact with aqueous solutions, and can absorb and retain significant volumes of fluid. Use of these dressings is growing at a double-digit rate, driven by an increasing elderly population afflicted with chronic wounds such as skin ulcers, due to diabetes, or pressure sores, resulting from being bedridden. These dressings are generally used to dress surface wounds, as opposed to cavity wounds because the hydrogel sheet materials do not possess the mechanical properties necessary to survive bending, folding and the torque necessary to pack a wound.

Hydrogel wound treatments have additionally been used as carriers for the delivery of therapeutic agents to a wound site, usually for the treatment of infection. Generally speaking, these hydrogels are the amorphous, or water-soluble type and these materials are in the form of a paste and packaged in a tube. For example, Intrasite gel, an amorphous hydrogel wound treatment manufactured by Smith & Nephew, is approved in the United Kingdom as a carrier for metronidazole for the treatment of fungating and other malodorous wounds. Generally, a medicament or drug used as the therapeutic agent is incorporated in the hydrogel during manufacture of the dressing, or, for film-type dressings, may be taken up into the polymer by swelling a dry film with an aqueous solution of the therapeutic agent. After the dressing is applied to the wound, the therapeutic agent diffuses into the tissue. It is expected that such therapies that combine treatment of wounds with moist wound dressing with delivery of a drug, especially an antibiotic, would provide a significant benefit to patients. Unfortunately, the use of hydrogels as carriers for therapeutic agents has been severely limited by the composition and resulting physical properties of available products. Many of the commercial moist wound dressings are composed of a crosslinked ethylene oxide polymer. These dressings are typically manufactured by irradiating an aqueous solution of a functionalized polyethylene oxide with ionizing radiation, resulting in a sheet of insoluble gel swollen with water. Any drug to be incorporated prior to the crosslinking step must be stable to this high-energy radiation. Alternately, it is possible to dehydrate the gel following crosslinking and rehydrate with an aqueous solution of the drug. However, dressings composed of polyethylene oxide frequently develop unacceptable cosmetic defects when dehydrated and rehydrated.

“Therapeutic agent,” as used herein, includes drugs and medicaments for treatment of pathological conditions and for prophylactic use. Included within the definition are antibacterial agents, inhibitors of enzyme function, anesthetics, peptides, growth factors, spermicides, antiviral agents, antifungal agents, antiparasitic agents, anti-inflammatory agents, antihistamines, analgesics, antineoplastic agents, hormones, kerolytic agents, tranquilizers, amino acids, vitamins, base-pair nucleotides and cytokines.

Polyanions, such as sulfonated styrene polymers, as class of compounds/molecules have been shown to exhibit potent antiviral and microbiocidal activity in vitro. In particular, polystyrene sodium sulfonate as well and sulfonated cyclodextrin have been shown to be 100% effective as a contraceptive agent in the rabbit by the inhibition of sperm hyaluronidase.

U.S. Pat. No. 5,840,387 to Berlowitz-Tarrant et al. discloses use of a sulfonated copolymer of styrene for delivery of therapeutic agents and U.S. Pat. No. 6,306,419 to Vachon et al. discloses the use of a sulfonated copolymer for use as a hydrogel wound dressing with controlled release capability. The entire contents of each are incorporated herein by reference. Therapeutic agents can be oligodynamic metals, such as silver as well as organic molecules, especially preferable are organic cationic molecules.

Chronic Wounds: A wound is a physical injury to tissue, or any degradation of its normal structure and function resulting from an internal or external pathology that results in an opening or break of the skin. A healing wound has aspects relating to control of infection, resolution of inflammation, angiogenesis, regeneration of a functional connective tissue matrix, contraction, resurfacing, differentiation, and remodeling. Chronic wounds are wounds that don't heal in a timely process.

An ulcer is described as a localized shedding of an epithelium. It is critical to treat such ulcers, because as soon as the epidermal cells die, a major barrier to bacteria is breached, and it can cause further necrosis to the surrounding tissues (Martini, 2001). An ulcer that is considered chronic, or nonhealing, is one that does not heal in a timely fashion.

There are many types of chronic ulcers, but the most common types that affect the skin are diabetic ulcers, venous leg ulcers, and pressure ulcers. These wounds can affect just the epidermis (partial-thickness), or they can reach into the dermis as well (full thickness). Pressure ulcers in the U.S. are estimated to occur in up to 2 million people (Kirsner et al., 1998), about 9.2% of all hospitalized patients resulting in a cost to the U.S. healthcare system of roughly $7 billion when all aspects of treatment including lost wages and travel are considered. These sores often occur when blood flow to an area of skin is cut off by continual pressure against superficial blood vessels. Diabetic foot ulcers affect 600-800 thousand people a year in the U.S., in about 6-20% of all diabetics hospitalized (Loots et al., 1999). Venous ulcers, mostly of the leg, affect 1 million people a year (Kirsner et al., 1998). These are mainly triggered by venous hypertension, corresponding to the failure of internal valves of the veins in the lower extremities. This situation may lead to neutrophil accumulation and activation in the tissue, causing the release of enzyme granules and free oxygen radicals that cause cell death and disruption of extracellular matrix (Smith et al., 2000). The leukocytes may also prevent the free flow of oxygen, nutrients, and cytokines by occluding the capillaries.

Chronic wounds represent a worldwide health problem that is growing largely as a result of increasing longevity of the American population. Pressure or decubitus ulcers represent an estimated 3% to 5% incidence in hospital patients. In patients with spinal chord injuries the incidence of chronic wounds is 25% to 85%.

The two very important enzymes associated with chronic wounds are the matrix metalloproteinase, MMP-8 which is collagenase and elastase, another very destructive enzyme. Both of these enzymes have been well characterized in non-healing wounds. An excessive concentration of both the serine protease elastase and matrix metalloproteinases (MMPs) in chronic non-healing wounds has been shown to render cytokine growth factors, fibronectin, and endogenous levels of protease inhibitors inactive. Although numerous studies with both animals and human beings have shown that growth factors may accelerate the healing of chronic wounds, therapeutic attempts have been largely unsuccessful.

The composition of a wound dressing, or packing, is relevant to designing a mechanism-based approach to protease inhibition in the environment of the wound fluid. (Wiseman D M, Rovee, D T, Alvarez 0 M Wound dressing: design and use in Wound Healing Biochemical & Clinical Aspects, eds. Cohen I K, Diegelmann, R F, Lindbald, W J, 1992, Hartcourt Brace Jovanovich, Inc. 562-580). The fiber or gel composition of synthetic dressings, applied to chronic wounds, include synthetic hydrogel polymers of the cross-linked and amorphous or water-soluble varieties, collagen, hydrocolloids, alginates and cotton and carboxymethylcellulose. Controlled release of agents linked with important roles in wound healing includes growth factors, antibiotics, and trace elements. The use of the enzyme inhibitor aprotinin for treatment of corneal ulcers was reported, however, there have been no known reports of treatment methods on the release of protease inhibitors into wounds.

U.S. Pat. No. 5,098,417 to Yamazaki et al. teaches the ionic bonding of physiologically active agents to cellulosic wound dressings.

U.S. Pat. No. 4,453,939 to Zimmerman et al. teaches the inclusion of aprotonin in compositions for “sealing and healing” of wounds.

U.S. Pat. No. 5,807,555 to Bonte et al. teaches the inclusion of inhibitors for alpha-1-protease, collagenase, and elastase in pharmaceutical compositions for promotion of collagen synthesis.

U.S. Pat. No. 5,696,101 to Wu et al. teaches use of oxidized cellulose (e.g. Oxycel) as a bactericide and hemostat in treatment of wounds.

World Patent WO 98/00180 to Watt et al. teaches complexation of oxidized cellulose with structural proteins (e.g. collagen) for chronic wound healing; and references the utility of oligosaccharide fragments produced by the breakdown of oxidized cellulose in vivo in the promotion of wound healing.

Many experts believe it logical to limit the area for drug treatment to the pelvic region for a number of gynecological indications. A variety of formulation and delivery technologies already exist to exploit the mucosal surfaces of the target area.

However, drug delivery using these existing formulations suffers from low levels of compliance due to difficulties of administration and, in some countries, a cultural resistance.

A particular advantage of using the vagina for drug delivery is the phenomenon known as the ‘first uterine pass effect’ caused by the significant number of blood vessels connecting the vagina to the uterus. Delivery of therapeutic agents via the vagina provides a preferential transfer to the uterus, thereby maximizing the desired effects while minimizing the potential for adverse systemic effects.

To date, vaginal delivery systems have been limited to vaginal rings for contraceptive use and suppositories for treatment of vulvovaginal infections. Vaginal rings have been the only long-term vaginal delivery systems commercially available. Variations of this device contain medroxyprogesterone acetate, estrogen, or progesterone dispersed throughout a matrix of polymerized silicone. The ring fits at the cervix and is utilized for contraception. Vaginal suppositories are routinely administered once a day and at bedtime since they inadvertently will leak. These devices have been modeled after rectal suppositories.

Vaginitis, vaginosis and other conditions caused by yeast, bacteria, viruses or parasites are common medical problems in women that are associated with substantial discomfort, particularly due to a copious pathologic discharge which is often accompanied by irritation, pruritus, odor or urinary symptoms. Several commonly known infections, such as yeast infection, bacterial vaginosis, trichomonas, chlamydia or gonococcal infections are common causes of the vaginal discharge.

Diseases Of The Vaginal Tract:

Bacterial Vaginosis: Bacterial vaginosis is the most common cause of vaginal discharge or malodor. It occurs when the normal flora of the vagina that produces Lactobacillus species is replaced with anaerobic bacteria. Bacterial vaginosis occurs more often in women who have multiple sexual partners, but it is not known if it is transmitted sexually.

All women with symptomatic disease require treatment, including those who are pregnant. Studies have shown that bacterial vaginosis is associated with preterm delivery in pregnant women who are already at high risk for preterm delivery. Bacterial vaginosis is also associated with pelvic inflammatory disease, endometritis and vaginal cuff cellulitis after invasive procedures.

A seven-day course of oral metronidazole (Flagyl) is recommended for the treatment of bacterial vaginosis. In addition, intravaginal clindamycin cream (Cleocin) and metronidazole gel (Metrogel) are recommended treatments in nonpregnant women.

Vulvovaginal Candidiasis: Symptoms of vulvovaginal candidiasis include pruritis, vaginal discharge and, sometimes, vaginal soreness, vulvar burning, dyspareunia and external dysuria. Vulvovaginal candidiasis can occur concomitantly with an STD or following antimicrobial therapy.

Several topical agents are still recommended for the treatment of vulvovaginal candidiasis and are first-line therapies in pregnant women.

Human Papillomavirus Infection: Human papillomavirus infection manifests as genital warts and is associated with cervical dysplasia. There are over 20 types of human papillomavirus, and not all types exhibit visible warts. Papanicolaou smears often identify associated cellular changes.

The goal of treatment is to eliminate visible genital warts. No evidence indicates that treatment affects the natural course of human papillomavirus infection or decreases its rate of sexual transmission. Two new treatments are available for patients' self-administration: podofilox (Condylox) and imiquimod (Aldara). Several factors should be considered when choosing a mode of therapy, such as wart size, wart number, anatomic site of wart, patient preference, cost of therapy, convenience, adverse effects and provider experience. Even with the patient-applied therapies, it is recommended that the health care provider apply the initial treatment to demonstrate the proper application technique.

Currently available treatments of vaginitis or other vaginal conditions include a systemic oral administration therapy or topically intravaginally introduced intravaginal creams, intravaginal suppositories, ointments or tablets which, in order to release the drug from these formulations, melt or dissolve in the vagina. The drug and other formulation components which are released during this process leak from the vagina creating unsanitary conditions and discomfort and also, more importantly, resulting in delivery of unpredictable amount of the drug.

One of the most recent studies, described in J. Reprod. Med., 44:543 (1999), reports that at this time, oral therapy is still preferred over intravaginal therapy. This is no doubt due to problems associated with vaginally delivered pharmaceutical agents. These problems include a discharge and leaking from the vagina which occurs during the treatment period, loss of drug due to such leaking, uncertainty of the amount of the drug delivered and general feeling of non-sanitary conditions which occur during such treatment.

Systemic treatment of vaginitis, which seems to be currently preferred, however, leads to the use of much higher doses of drugs which are potentially dangerous and typically cause severe secondary symptoms and complications. For example, local treatment of vaginal candiditis, a yeast infection, requires the use of antifungal drugs, such as nystatin, clotrimazole, miconazole and such similar drugs, administered as a cream via applicator, as suppository, or as a tablet, at bedtime. Due to a leakage encountered with such local treatment, once a day at bedtime treatment is recommended.

Once-a-day local administration of the drug does not provide continuous level of drug to treat the vaginal conditions, to deliver the drug to the uterus or to the general blood circulation and may lead to development of drug-resistance.

Thus it would be advantageous to have available treatment which would provide a continuous and predictable delivery of the drug to the vaginal mucosa and/or which would deliver the drug transvaginally into uterus or to the general blood circulation to avoid a necessity to administer the drug in high doses and to avoid a deactivation of the drug by the gastrointestinal tract.

Bioadhesive polymers can aid with the absorption of drugs through mucosal surfaces. Bioadhesive polymers can be used for almost any region that you have epithelial cells, including oral, buccal (cheek), GI tract, rectal, or vaginal delivery. Adhesive molecules bring the delivery system closer to the mucosa. To accomplish this improved delivery, some groups have designed polymers with a high amount of carboxylic acid, which hydrogen bonds with the carboxylic acids in epithelial cells. Sulfonic acid polymers, such as a sulfonated styrene polymer will also hydrogen bond with the carboxyl groups of epithelial cells thus bringing delivery closer to the mucosa.

Transvaginal delivery of a drug via a vaginal device has been disclosed by inventors and is described in the U.S. Pat. Nos. 6,416,779, 6,197,327 and in U.S. Pat. No. 6,086,909, all of which are incorporated herein by reference.

It is therefore one objective of this invention to provide a device, composition and a method for topical and local treatment of vaginal infections by providing an intravaginal device comprising an antifungal, antibacterial, antiviral, trichomonicidal or parasiticidal agents incorporated within the device. The method of the invention provides a treatment of vaginal candidiasis, bacterial vaginosis, genital herpes, chlamydiosis, trichomoniasis, gonorrhea and human papilloma virus which eliminates the need for systemic treatment, which permits continuous delivery of the drug to the vaginal mucosa locally and topically and, where appropriate, which permits transvaginal delivery of the drug to the uterus and/or to the general circulation.

Prior treatments have been attempted rectally using suppositories and enemas. Rectal administration, while often more effective than oral administration, is limited in that most rectally administrable dosage forms are capable of producing the intended result only in the immediate area, not reaching the upper portions of the colon. This is because the length of the colon reached is volume dependent, usually reaching only as far as the splenic flexure. In addition, rectal administration is messy and inconvenient, as well as not readily acceptable to the general patient population. Furthermore, if the patient suffers from severe inflammation of the rectum, he may experience difficulty with retention enemas.

Thus, an orally administrable dosage form to treat colonic diseases would usually be preferred and is often required. Orally administrable treatments, using tablets, capsules, and the like, have been attempted. However, to reach the colon intact, the dosage form must withstand the rigors of the transit through the gastro-intestinal tract. These rigors include at least a million-fold variation in hydrogen ion concentration, wide variations in osmotic pressure from the surrounding fluids, a variety of enzymes, and a strong mechanical grinding force.

Furthermore, most of these orally administered dosage forms result in delivery of the drug in the upper portion of the gastro-intestinal tract or, in the case of controlled release dosage forms, deliver drug throughout the entire length of the gastrointestinal tract instead of concentrating delivery primarily within the colon. Thus, in either case, by the time the dosage form reaches the colon, the drug concentration is diminished or even depleted. In addition, the acidic and enzymatic environment of the stomach may inactivate a substantial mount of the drug, particularly protein or peptide-like drugs. Even if the drug is released from the stomach in its active state, such drugs frequently are metabolized or inactivated in the small intestine. Thus, little if any of the drug from these conventional dosage forms is available for producing a therapeutic result in the colon, especially if the dosage form reaches the colon essentially devoid of drug.

Drug delivery to the colon is difficult not only for the above-mentioned facts, but also because of the uncertainty of the transit time from oral ingestion to arrival at this pre-selected site. The time of retention within the stomach is most variable, depending both on the size of the dosage form and the mount of food present at the time of ingestion. The drug delivery device may remain within the stomach from about 0.5 to about ten hours. The device then enters the small intestine where retention time is significantly more constant and less dependent upon the mount of food present. It takes from about three to about six hours to travel the length of the small intestine to the beginning of the colon. The device may then remain within the colon from about ten to about fourteen hours in a subject with normal motility.

Thus, the time span necessary to delay release of the drug from an orally administered dosage form until the beginning of the colon is wide. However, the time span can be considerably narrowed by measuring the time from arrival in the small intestine instead of from the time of ingestion. Drug delivery in the stomach may be prevented by the use of an enteric coating which is resistant to the gastric fluids. As such a coating is not soluble in fluids with an acidic pH, such as that of the stomach, application to the outside of the dosage form inhibits release prior to reaching the higher pH of the small intestine. Once the dosage form reaches the small intestine and the enteric coating dissolves, drug release needs to be delayed only an additional three to six hours to result in substantially no active agent being delivered before the colon.

Although some drug may reach the colon passively, conventional peroral dosage forms are not designed to deliver their contents specifically to the colon. Generally, they are formulated to be immediate release devices which disintegrate in the stomach, duodenum, or small intestine, allowing the drug to be immediately exposed to the local environment.

Controlled release dosage forms, for example Orally Releasing Osmotic Systems or OROS.RTM. (Alza Corporation), have been developed (U.S. Pat. No. 3,845,770). Although the benefits of controlled release are significant, such as reduction in the number of doses and steady drug levels in the blood, they are generally no more effective than conventional tablets in delivering the active agent primarily to the colon.

Several delivery forms have been developed which attempt to deliver active agent primarily to the colon. These methods rely upon either the environmental conditions surrounding the system, particularly pH, bacterial count and/or time.

Wong, et at. (U.S. Pat. Nos. 4,627,851; 4,693,895; and 4,705,515) disclose a tri-laminated core in which the first layer is composed of an insoluble, but semi-permeable composition, the second is a microporous combination of water insoluble polymer and osmotic solute, and the third contains an enteric composition. This dosage form has a delayed onset of delivery for a period of about two hours after it exits the stomach, after which only about 50% of the drug is released within twenty-four hours. This drug delivery time scheme is insufficient to insure that the bulk of the drug is delivered to the colon.

Theeuwes, et al. (U.S. Pat. No. 4,904,474) disclose a dosage form which has a two-layered internal compartment with a first layer of the drug in an excipient layer adjacent to an exit passageway and a second layer of a push component. The internal compartment is surrounded by a semi-permeable wall and then an enteric layer. Theeuwes's dosage form results in a delay of the onset of delivery in intestinal fluid for a period of about two hours. This represents a delay period too short, and a delivery rate too slow to insure the bulk of the drug is delivered to the colon.

Ring, et at. (WO 91/07949) disclose a tablet core coated with two laminates. The outer laminate is an erodible acrylic polymer and the inner laminate consists primarily of amylose in the glassy state which can only be degraded in the presence of fecal microflorae.

The instant parametric drug delivery devices can also be used to deliver a drug intermittently at pre-selected times such that the patient receives the drug when needed. This is of particular importance in treating diseases which have symptoms which do not remain constant throughout the day and night.

For example, blood pressure is known to follow a circadian rhythm during a 24-hour period. In some subjects the highest pressure occurs in the morning shortly after the individual awakes, suggesting that it would be appropriate to deliver an antihypertensive agent such as a beta-blocker to such a patient sufficiently before awakening so as to mitigate the effects of the disease at the most appropriate time interval. In order to accomplish this without disturbing the patient's sleep, it is necessary to administer the drug in the evening in a form that is activated just before the patient arises.

Savastano et al. (U.S. Pat. No. 5,681,584) describe a targeted controlled release device that delivers a pharmaceutical agent to the colon via the rectum.

Another example is the treatment of asthma with the agent theophylline. The drug has a rather narrow therapeutic index with minimum effective blood concentrations of 6-10.mu.g/ml and toxic levels of approximately 20.mu.g/ml. However, the serum theophylline concentrations required to produce maximum physiological benefit may fluctuate with the degree of bronchospasm present and are variable. Asthma often exhibits more serious symptoms in the evening, while theophylline absorption may change due to posture and changes in the circadian rhythm. This suggests that the nighttime dosing need not be identical to the daytime dosing regimen, and it is recommended that the extended release formulation not be given in the evening. Thus, a sustained acting dosage form for the day, with a bolus dose of theophylline at bedtime combined into a single peroral drug delivery system requiring once per day dosing in the evening is of possible benefit.

Many controlled release dosage forms are created by the use of special water insoluble membranes which either limit the flow of gastrointestinal juices into the system, or modulate the release of dissolved substances out of the system. Application of such a membrane was initially accomplished by thin layer, spray application of lacquer coatings made with organic solvents. These processes allowed the manufacturer to achieve the desired membrane qualities in short time using few components. However, it was eventually realized that the processes were often dangerous in that excessive use of organic solvents were capable of causing irreversible harm to the environment and produced dosage forms which contained extraneous, undesirable residuals.

Whenever organic solvent is used in a pharmaceutical process, measures need to be taken to protect the operators who produce the dosage forms and the environment from overexposure to the hazardous, often teratogenic and carcinogenic materials. Additional precautions are necessary to protect personnel, equipment and facilities from harm due to the ignition of explosive vapors. Even if these immediate problems can be solved through engineering means, it is still possible for detectable levels of residual solvent to remain in the finished dosage form, the long-term effects of which are either undesirable or not yet established.

Several manufacturers of coating equipment responded to the challenge of minimizing the dangers of using hazardous solvents by building machines which contained and controlled the exhaust vapors from organic solvent coating processes. Despite the capability of these machines to minimize the problems of explosion and exposure hazards, the equipment is complicated, costly to operate, and requires rather expensive maintenance even on a murine basis. It also does not address the problem of residual solvent remaining in the finished dosage form. This is ameliorated by storing the coated tablets in containers at high temperatures and humidities in order to draw the solvent out of the tablets; however, solvent extraction from finished dosage forms adds costs to the manufacturing process in additional capital equipment expenditures, processing time and analytical requirements.

The impetus for seeking new manufacturing techniques is obvious. The U.S. Food and Drug Administration and Environmental Protection Agency are continuously urging all manufacturers to reduce, and wherever possible, to eliminate the use of organic solvents in manufacturing.

Rather than pursuing costly engineering solutions to the problem, raw material suppliers were encouraged to develop aqueous dispersions of the materials most frequently employed to produce film coatings for tablets, pellets and particulate dosage forms. Aqueous dispersions allow utilization of existing equipment and familiar processes, thus avoiding the expenses of capital investments, maintenance, process validation and retraining of personnel.

All references, patents and patent applications cited herein are hereby incorporated by reference in their entirety.

### SUMMARY OF THE INVENTION

In one aspect, the present invention relates to a method for inhibiting elastase and/or collagenase in a wound, including contacting the wound with a composition including a combination of a sulfonated styrene copolymer and a tetracycline, especially doxycycline.

In another aspect, the present invention relates to a method for inhibiting elastase in a wound including contacting the wound with a composition including a sulfonated styrene copolymer in salt form; the composition may additionally include a tetracycline. In either of these methods, the composition may be disposed on a surface of a wound dressing, and the wound dressing may include a substrate selected from a foam, a woven fabric, a knit fabric, and a nonwoven fabric.

In another aspect, the present invention relates to a composition including a combination of a sulfonated styrene copolymer and a tetracycline, especially doxycycline. In these compositions, at least a portion of the sulfonated styrene copolymer may be in the form of a salt, especially an ammonium salt.

In yet another aspect, the present invention relates to a composition including a combination of a sulfonated styrene copolymer and an amino acid, especially proline or arginine.

In yet another aspect, the present invention relates to a process for manufacturing articles composed of at least one sulfonated styrene copolymer, said article selected from tubes, sheets and 3-D constructs, including electrodepositing the sulfonated styrene polymer to form the article. The 3-D constructs and/or tubes may be used in vascular grafts.

In yet another aspect, the present invention relates to a method for treating a vaginal infection, including incorporating a sulfonated styrene polymer into a tampon, and contacting the vaginal wall with the tampon. The sulfonated styrene polymer includes a therapeutic agent for treatment of the infection. The sulfonated styrene polymer may be incorporated into the tampon by coating the tampon with the polymer.

In yet another aspect, the present invention relates to a method for delivering a therapeutic agent to a colon of a mammal, including incorporating a sulfonated styrene polymer into a suppository and contacting the wall of the colon or rectum of the mammal with the suppository. The sulfonated styrene polymer includes a therapeutic agent for delivery to the colon.

In yet another aspect, the present invention relates to a method for controlling biological organisms on a porous surface, including forming a coating, composed of an ammonium salt of a sulfonated styrene polymer, on the porous surface. This may be accomplished by coating the porous surface with the sulfonated styrene polymer in acid form and converting the acid form to the ammonium salt form. The porous surface may be fabric or paper, especially an article selected from a garment, an air filter, a gas filter, a laboratory work surface, or laboratory wipe.

In any or all of the above methods and composition of the present invention, the styrene sulfonate copolymer may include residues derived from an olefin comonomer. The olefin comonomer may be selected from ethylene, butylene, isobutylene, butadiene, isoprene and combination thereof. The sulfonated styrene copolymer may be a block copolymer, particularly, a sulfonated styrene-ethylene-butylene-styrene triblock copolymer.

### BRIEF DESCRIPTION OF THE DRAWINGS

FIG. 1 is a cross sectional view of both a sulfonated styrene polymer coated sheet substrate for use as a wound dressing;

FIG. 2 is a cross sectional view of a sulfonated styrene polymer coated substrate in the form of a cavity insert, tampon, or section of rope;

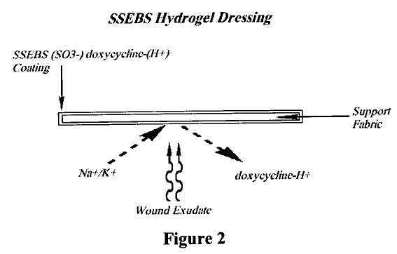

FIG. 3 is an example of a sulfonated styrene polymer coated coronary stent containing bound gentamicin; and

FIG. 4. is an example of a drug release profile as measured using UV spectroscopy. More specifically, FIG. 4 is representative of the release of the drug tetracycline (into Tris buffer) from a 30% sulfonated SEBS polymer.

### DETAILED DESCRIPTION OF THE INVENTION

It has been surprisingly discovered that sulfonated styrene polymers are useful as a hydrogel material that can be used to prepare lacquers or latexes, with or without therapeutic agents, for coating onto other material substrates to yield medical articles useful for treating medical conditions. In particular, medical articles such as hydrogel wound dressings and inserts for cavities created by surgery or those cavities common and natural in mammalian anatomy. The term “sulfonated styrene polymer” as used herein refers to a polymer having residues derived from a styrene monomer, the aromatic ring of which is substituted with at least one sulfonate group. The term encompasses homopolymers containing residues derivable from styrene sulfonate, and copolymers containing residues derivable from styrene and styrene sulfonate, as well as copolymers containing residues of other comonomers in addition to styrene and styrene sulfonate. These hydrogel polymers do not require chemical crosslinking, are soluble in common solvents and can be dehydrated and re-hydrated without the formation of cosmetic defects.

These sulfonated styrene polymeric hydrogels are unique, and given their superior properties relative to chemically cross-linked materials, excellent candidates for use in wound care and other medical applications for at least two very important reasons. The first is related to processing advantages that these materials possess. Sulfonated copolymer hydrogels, such as sulfonated styrene-ethylene-butylene-styrene, sulfonated styrene-ethylene and other copolymers such as sulfonated SIBS, SEPS and SIS are soluble in common organic solvents such as tetrahydrofuran, chloroform, dichloro-methane, and methyl-ethyl ketone. As such, high solids lacquers are easily prepared allowing for the casting of films, coating of articles, and impregnation of fabrics using dipping, painting, or spraying. These sulfonated hydrogels may also be processed to yield latex formulations, thus eliminating the use of organic solvents.

Furthermore, sulfonated block copolymer hydrogels may be used in their acid form (—SO₃H), or as the respective salt following deprotonation by base. Counterions include sodium, lithium, potassium, calcium, manganese, magnesium, silver, gold, ammonium (NH₄⁺) and primary (NH₃R⁺), secondary (NH₂R₂⁺), tertiary (NHR₃⁺) and quaternary (NR₄⁺) ammonium, as well as organic cations derived from therapeutic agents such as antibiotics or amino acids. The (—SO₃⁻) group is a strong binder of Zn²⁺, and given the lamellar structure and the high ion conductivity of SSEBS, as a result of organized (—SO₃) channels, the polyanionic sulfonated styrene polymer is conformationally arranged to disrupt the active (Zn⁺⁺) binding site of matrix metalloproteinases via the complexation of zinc ion. Moreover, the bulk polyanionic character of the deprotonated sulfonated styrene polymer is favorable for the electrostatic sequestering of elastase from wound fluid.

These sulfonated copolymer hydrogels have chemical structures that allow them to be processed from a solution/lacquer using electrodeposition or electroprocessing. Using this technique, ultrathin fibrous, high surface area device configurations may be created. Devices in the forms of sheets, tubes or other configurations including pouches or spheres may be created. The electroprocessing technique may be carried out with therapeutic agents, including biomolecules, included in the lacquer from which the polymer is spun to create drug delivering polymer strands/fibers. However, in order to fabricate sulfonated styrene polymer hydrogels that incorporate biomolecules, it may be necessary to hydrate the styrene copolymer hydrogel in the presence of an aqueous solution of the biomolecule of interest in order to avoid denaturation of the protein, peptide or the like. However, small, typically synthetic species such as steroids (dexamethasone), antibiotics (tetracyclines/doxycycline, gentamicin), and antineoplastic agents such as paclitaxel or sirolimus may be incorporated into the organic (solvent) solutions of the hydrogel of interest and dip coated, sprayed, painted, or electroprocessed in a straightforward manner. Furthermore, the robustness of these materials allows for them to be press-formed using high pressure into sheet, tube, and other pertinent forms.

Furthermore, these sulfonated styrene polymeric hydrogel materials do not require chemical or radiation crosslinking in order to render them with mechanical properties and characteristics suitable for them to be used in a medical application. Chemical and/or radiation crosslinkable hydrogels, such as poly (vinylpyrrolidinone) or polyethylene oxide, have poor mechanical properties even after crosslinking, thus limiting their applicability in medical articles. For example, when used as wound care materials, chemically cross-linked hydrogels are formed into sheets/films for application as a topical wound dressing product. By virtue of the poor mechanical properties of these materials, they cannot be formed into dressings with the versatility to be used as wound coverings or as wound packing(s), either as free-standing films or as gauze or fabric/material-supported configurations.

Sulfonated styrene polymers: The sulfonated styrene polymer may include residues derived from at least one olefin comonomer in addition to the residues derived from styrene. The olefin comonomer is preferably ethylene, propylene, butylene, isobutylene, butadiene or isoprene, or a combination of two or more of these. Preferred sulfonated styrene polymers are sulfonated styrene-ethylene-butylene-styrene triblock copolymers, sulfonated reduced statistical styrene butadiene copolymers and sulfonated statistical styrene ethylene copolymers. The term “reduced” is used herein to designate a copolymer that has been hydrogenated in order to reduce residual double bonds, prior to the sulfonation step. The term “statistical” refers to copolymers that are synthesized by methods that are not designed to produce blocks or grafts in the copolymer; these polymers are also commonly referred to as random copolymers.

The sulfonated styrene polymer preferably comprises from 20 to 80% styrene, and preferably has a molecular weight of at least 20,000. At least 15 mole percent of the residues derived from styrene are sulfonated, and more preferably at least 30 mole percent of the styrene residues are sulfonated.

The sulfonated styrene polymers useful as the wound dressings of the present invention or as the coatings for medical devices of the present invention are hydrophilic, hydrogel-type materials that can absorb and retain a relatively large amount of water or water-containing fluid. In addition, the sulfonated styrene polymers possess good mechanical strength and abrasion resistance when swelled with water, without requiring crosslinking, such that a moist wound dressing containing the sulfonated styrene polymer maintains its integrity without disintegrating. Sulfonated styrene polymers provide a convenient and effective means to deliver therapeutic agents, particularly silver ion, to tissues in contact with the copolymer.

The composition of sulfonated styrene polymers useful for wound dressings, or as coatings for medical devices, typically ranges from about 20% styrene to about 80% styrene. That is, the polymer contains about 20-80% by weight of residues derived from styrene before sulfonation of the aromatic ring of the styrene residues. Homopolymers of styrene may be sulfonated to produce a copolymer containing residues derivable from styrene and styrene sulfonate. The sulfonated styrene polymer may additionally comprise residues derived from at least one olefin comonomer. Preferred olefin comonomers include monoolefins, such as ethylene, propylene, butylene, and isobutylene, and also diolefin monomers, such as butadiene and isoprene. Other comonomers, such as acrylate monomers, may be used, provided that the properties of the copolymer are sufficient for use as a wound dressing. The composition may be adjusted by varying the level of styrene and/or the comonomers(s) to provide desired properties in the end product. Properties that are significant for application as a moist wound dressing are tensile strength, abrasion resistance, compliance, hydrophilicity (water uptake), and biocompatibility. In order to be useful, the dressing should preferably be strong, elastic, highly conformable, inexpensive, absorbent, and sterilizable. Level of sulfonation largely determines the maximum amount of water taken up by the polymer.

The sulfonated styrene polymer may also be blended with other polymers and used for preparing lacquers for dipping, painting, spraying, electrospraying, or electroaerosoling. These blends, depending on the amount of each polymer and the thermodynamics of mixing, can afford materials ranging from phase-separated to single phase. An advantage of blending is that selected properties of the individual components may be obtained in the resulting material. Block copolymers having both components of the blend in a single chain may be used to the increase the compatibility of the blend components. It should be noted that blending is not limited to polymer pairs, and thus three-component and higher mixtures are possible.

The preferred level of sulfonation of the styrene residues is at least 15 mole percent, and is preferably at least 30 mole percent. However, where a blend of a styrene/styrene sulfonate copolymer with another polymer is utilized, higher levels of styrene sulfonate may be desirable. Sulfonation of the styrene residues is typically performed after completion of the polymerization. Methods for sulfonating styrene copolymers are known in the art. One suitable method is described in U.S. Pat. No. 5,468,574 to Ehrenberg et al. Therein, sulfur trioxide and triethyl phosphate in a solution of methylene chloride/cyclohexane are used as sulfonating agents for a styrene-ethylene-butylene-styrene block copolymer. Sulfonation of hydrogenated block copolymers of styrene and butadiene to a level of about 25 mole percent is known in the art as described in U.S. Pat. No. 5,239,010 to Balas et al. A preferred method of sulfonating at the aromatic ring of the styrene residues, whereby high levels of sulfonation may be achieved, is described in published PCT application, WO 99/38896. The application discloses the preparation of an acetyl sulfate sulfonation agent by the addition of sulfuric acid to a solution of acetic anhydride in 1,2-dichloroethane (DCE). An appropriate amount of the sulfonation agent is reacted with a styrene copolymer in a DCE solution to yield a copolymer sulfonated to a desired level, up to about 80 mol %.

When an unsulfonated styrene copolymer contains residues derived from a diolefin comonomer, such as butadiene, residual alkene functionality is present in the copolymer. In this case, the copolymer may be hydrogenated in order to reduce the double bonds prior to the sulfonation step. The resulting copolymer is referred to as a reduced or hydrogenated copolymer. The copolymer may be hydrogenated by methods known in the art, for example, by hydrogen gas in the presence of catalysts such as Raney Nickel, platinum or palladium. Hydrogenated statistical copolymers of styrene and butadiene are also commercially available.

Several types of styrene-containing polymers are commercially available, including statistical, block and graft copolymers, and combinations of these types. The term “statistical” is well known in the art, and refers to copolymers that are synthesized by methods that are not designed to produce blocks or grafts in the copolymer. (This type of polymer is also commonly referred to as a random copolymer.) The monomers polymerize according to their relative reactivities. Any of these types may be used in the methods and compositions of the present invention, including sulfonated styrene-isoprene-styrene block copolymers (SIS), hydrogenated SIS block copolymers including sulfonated styrene-ethylene-propylene-styrene block copolymers (SEPS) and sulfonated styrene-ethylene-ethylene-propylene-styrene block copolymers (SEEPS), sulfonated styrene-isobutylene-styrene block copolymers. Particularly preferred sulfonated styrene polymers are sulfonated styrene-ethylene-butylene-styrene triblock copolymers, statistical sulfonated styrene butadiene copolymers and sulfonated statistical styrene ethylene copolymers.

Unsulfonated styrene-ethylene-butylene-styrene triblock copolymers may be obtained from Kraton Polymers as the Kraton series of polymers. The styrene content of the Kraton copolymers is typically about 30% before sulfonation. Similar materials are also available from Kuraray. Unsulfonated rubbery styrene butadiene copolymers, known as styrene butadiene rubber (SBR) are commercially available from Kraton Polymers, Kuraray, and Goodyear.

Molecular weight of the polymer preferably ranges from about 20,000 to about 1,000,000, and more preferably from about 50,000 to about 900,000. With regard to a lower limit for molecular weight, highly sulfonated styrene polymers having a relatively low molecular weight may have some solubility in water limiting the use of the material in a variety of medical products including wound dressings, medical device coatings such as medical stents, pharmaceuticals such as for enteric drug delivery, vaginal and rectal inserts for the delivery of a variety of therapeutics such as antibiotics or antifungal agents for the treatment of a variety of medical conditions, coated release articles for implantation at the site of a tumor for the purposes of targeted delivery of antitumor agents, garments, air and gas filters, and laboratory work surfaces where the control of biological organisms may be desirable. An example being the coating of fabric or a high surface area filter with SSEBS and its subsequent conversion to the NH₄⁺, or the NR₄⁺ salt which have been shown to be bactericidal, and cytotoxic.

In general, it is desirable that the wound dressings of the present invention contain a matrix of a styrene sulfonate hydrogel polymer having a molecular weight sufficiently high that the polymer is not water-soluble. Since the preferred level of sulfonation is at least 15% mole percent, molecular weight is preferably at least 20,000. Sulfonated styrene polymers, due to the lack of chemical crosslinks, are typically soluble in common organic solvents. For example, sulfonated SEBS is soluble in tetrahydrofuran. Copolymer solutions are advantageously used in manufacturing the wound dressings and coated implantable medical devices of the present invention, and to incorporate therapeutic agents in the same. With regard to an upper limit for molecular weight, in order to control the viscosity of sulfonated styrene polymer solutions during the manufacturing process, it may be desirable to limit the molecular weight of the copolymer to less than about 1,000,000.

A particular advantage of using sulfonated styrene polymers as a hydrogel-type material in a wound dressing or as a coating for implantable medical devices is that therapeutic agents may be conveniently incorporated in the copolymer. Because sulfonated styrene polymers are soluble in some common organic solvents, a solution of a sulfonated styrene polymer in a suitable organic solvent may be combined with a solution of a therapeutic agent in a compatible solvent. Alternatively, because films of sulfonated styrene polymers may be rehydrated without cosmetic defect, water-soluble therapeutic agents may be incorporated in the copolymers by swelling the dehydrated sulfonated styrene polymer dressing with an aqueous solution of one or more therapeutic agents.

A wound dressing of the present invention, containing a sulfonated styrene polymer, may be fabricated in any convenient form. Preferably, it is fabricated as a substrate having a sulfonated styrene polymer applied thereto or as a laminate having a layer containing a sulfonated styrene polymer. A sulfonated styrene polymer may be applied to a substrate by impregnating, coating and/or encapsulating the same with a sulfonated styrene polymer. Exemplary materials that may be suitable as substrates include porous knitted, woven or nonwoven manmade or natural fiber-based fabrics. The fabrics may be composed of cotton, wool, rayon, polyamide, polyimide, polypropylene, or polyester fibers. The wound dressing may be secured to the wound by any suitable means, such as tape or wrapping with a fabric strip.

A wound dressing in the form of a laminate is typically composed of a backing, which is optionally coated with an adhesive layer, and a layer containing a sulfonated styrene polymer. The backing may be a solid film, a perforated film, a woven fabric, a nonwoven fabric, a knit fabric, or a laminate of fabrics and/or films. Adhesives suitable for medical use are preferred. The adhesive layer serves to attach the copolymer to the backing, and/or to affix the dressing to the wound or to the skin near the wound. The backing, or the adhesive layer, if an adhesive layer is used, is partially or completely covered with a layer containing the sulfonated styrene polymer. This layer forms the surface that may be placed in contact with the wound during treatment. This layer may be composed of a sulfonated styrene polymer alone, that is, as a film or coating, or of a substrate impregnated, coated and/or encapsulated with the copolymer.

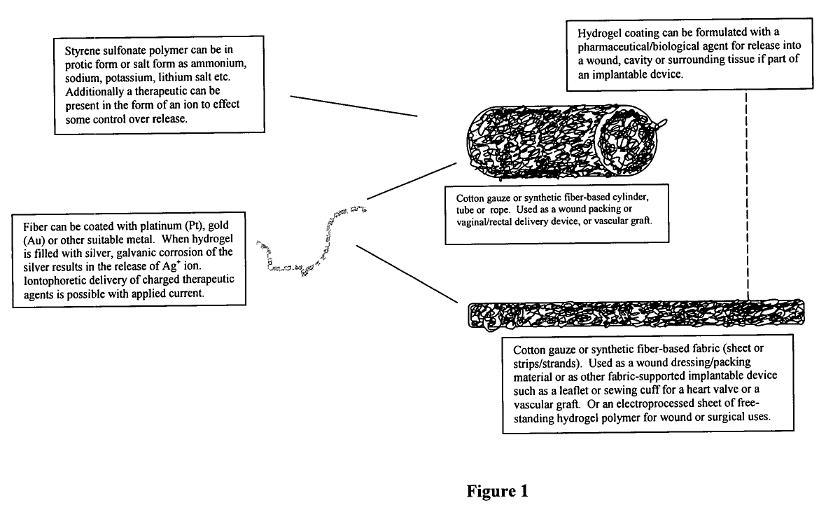

A wound packing of the present invention, containing a sulfonated styrene polymer, may be fabricated in any convenient form. Preferably, it is fabricated as a substrate having a sulfonated styrene polymer applied thereto having a layer containing a sulfonated styrene polymer. A sulfonated styrene polymer may be applied to a substrate by impregnating, coating and/or encapsulating the same with a sulfonated styrene polymer. Exemplary materials that may be suitable as substrates include porous knitted, woven or nonwoven manmade or natural fiber-based fabrics. The fabrics may be composed of cotton, wool, rayon, polyamide, polyimide, polypropylene, or polyester fibers. The wound dressing may be secured to the wound by any suitable means, such as tape or wrapping with a fabric strip. A wound dressing for packing a wound can be in the form of sulfonated styrene polymer coated gauze, or in the form of a rope-like substrate. A wound packing device in the form of coated fabric (sheet), stranded sheet, or rope need not contain a backing material. FIG. 1 provides an example of a cavity packing wound treatment.

Drug delivery articles for placement into an orifice, cavity or surgically created space comprise a first layer having a first surface which is contactable with the wound, cavity, or orifice and has disposed thereon a sulfonated styrene polymer. The drug delivery article may be in the form of a tampon, fiber, wafer or other suitable form. The sulfonated styrene polymer is furthermore formulated to include a therapeutic agent either by ion exchange, i.e. after fabrication of the device by aqueous uptake of the therapeutic agent, or inclusion in one step from the coating solution of the sulfonated styrene polymer.

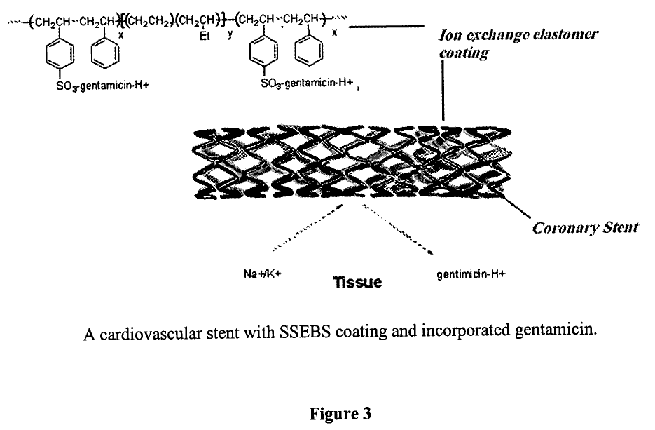

Sulfonated styrene polymers containing a therapeutic agent may also be used to coat medical devices for implantation in the body. The therapeutic agents may be chosen in order to prevent infection, prevent tissue proliferation, or minimize inflammation. The therapeutic agent is chosen for a specific action and expected outcome and may be chosen by someone skilled in the art of medical device development. Implantable medical devices that may be coated with a sulfonated styrene polymer containing a therapeutic agent are those that come into contact with a body fluid or tissue for a period of time whereby microorganism proliferation on the surface of the device is a concern, or tissue overgrowth and/or inflammation as a result of healing following surgical injury, or the stimulation of new blood vessel growth is desirable. These include, but are not limited to stents, catheters, cannulae, vascular grafts, artificial hearts, heart valves, venous valves, pacemakers and leads therefor, implantable defibrillators and leads therefor, orthopedic pins and plates, artificial joints, prostheses, tracheal tubes, ventilator tubes, insulin pumps, biosensors, wound closure devices, hemostats, drains, shunts, connectors and those other medical devices typically used in an environment where it is desirable to prevent or stimulate a biological response. FIG. 3 details a stent with a coating of gentamicin for preventing infection. The substitution of paclitaxel for gentamicin may be carried out in a straightforward manner yielding a diffusion controlled release device resulting from a lack of ionic interaction between the polymer and the therapeutic agent. However, it is important to note that therapeutic agents that contain hydroxyl moieties can esterify the sulfonated styrene polymer to yield sulfonated styrene polymer covalently functionalized with a drug. In the case of sulfonate esters, hydrolysis occurs readily, thus liberating the therapeutic agent. Stoy et al. disclose the acid hydrolysis of polyacrylonitrile using nitric acid (U.S. Pat. No. 3,897,382), as well as basic hydrolysis of polyacrylonitrile using alkali base such as sodium hydroxide and sodium isothiocyanate (U.S. Pat. No. 6,232,406).

It is understood in the art that nitrile groups, in particular, can be converted to amide or carboxyl groups by the action of acid hydrolysis. HYPAN® hydrogel polymers are based upon hydrolyzed polyacrylonitrile polymers. These materials have unique and interesting properties however, these materials do not have the structural characteristics that a styrene based copolymer would have. Thus, SAN or ABS copolymers could be rendered hydrophilic by the same action resulting in virtually crosslinked hydrogels with amide or carboxy or imine or amidine groups and these materials will have greatly enhanced mechanical properties relative to HYPAN® polymers. The conversion of nitrile (—CN) to a variety of groups (is well understood by those skilled in the art of organic chemistry and as such the groups listed above represent a small number of the possibilities.

The hydrophilic styrene-containing copolymers comprising this inclusive group may have hydrophilic functional groups adjoined to the styrene aromatic ring, or adjoined to at least one of the co-monomer units.

Additionally, any of the above listed styrenic copolymers may be hydrogenated in order to remove residual unsaturation. This hydrogenation may be carried out prior to the chemistry required for addition of, or conversion from one functional group form to another functional group form thus rendering the resultant material hydrophilic.

Styrene-acrylamide and or styrene-acrylic acid copolymers resultant from the hydrolysis of SAN, or styrene-butadiene-acrylamide and styrene-butadiene-acrylic acid, which are the result of the hydrolysis of acrylonitrile-butadiene-styrene, are expected to have excellent solubility in organic solvents such as dichloromethane, THF, or other halogenated and/or polar solvents as a consequence of the presence of styrene and/or butadiene (EB phase) in the polymer backbone. The solubility of these (functionalized) co-polymers is attributed to the chemical structure and make-up of these materials. Generally, their copolymeric nature, i.e. their possessing of for example poly (styrene) segments and poly (butadiene) segments (as in the case of styrene-butadiene copolymers), or hydrolyzed styrene-acrylonitrile (SAN) copolymers, i.e. where the hydrolyzed nitrile moieties result in/yield amides i.e. poly (acrylamide) segments and thus yield poly (styrene-acrylamide) copolymers. Similarly, the hydrolysis of acrylonitrile-butadiene-styrene (ABS) copolymers, i.e. where the hydrolysis of the nitrile moieties result in/yields amides or more correctly poly (acrylamide) segments, thus result in acrylamide-butadiene-styrene copolymers, provide these materials with their excellent mechanical and processing properties that include processing from solvent by dipping, painting, coating, spraying, electroprocessing or press forming. The association of the hydrophobic (hydrocarbon) segments within these polymers (i.e. butadiene and styrene, as depicted in cartoon FIG. 1) is ultimately responsible for the excellent mechanical properties of these materials. All of these styrene/polystyrene copolymeric hydrogel materials mentioned herein may be blended with other appropriate and biocompatible polymeric materials in order to yield composite devices that are formed using dipping, spraying, painting, electroprocessing, or press forming. Thus it is the premise of this application that hydrogels based on styrene copolymers, as a class of materials, will possess two very important characteristics: 1. solubility in common organic solvents, and 2. excellent mechanical properties, as compared to well-known cross linked hydrogel systems, as a consequence of the block copolymer, and microphase separated nature of these materials.

The sulfonic acid on the polymer may be converted to the ester form in the presence of an alcohol. The esterifying alcohol may be chosen from a group of alcohols that have therapeutic benefit. Because sulfonic acid esters are hydrolytically unstable, we anticipate that the alcohol (therapeutic) portion of the ester may be liberated in the presence of water/body fluids following implantation.

Thus, because many therapeutic agents contain OH groups, they may be used to esterify the polymer thus allowing for the liberation of the therapeutic agent when placed in contact with an aqueous environment. This approach to controlled release may be utilized for devices such as stents, vascular grafts and other devices where exacerbated responses to the implant threaten lifetime or patient outcomes. Additionally, the prodrug may be implanted into, or near a tumour such as a glioma or otherwise in order to deliver an antineoplastic or other OH containing therapeutic agent over time.

The present invention also relates to a wound dressing for covering or packing a wound, or a drug delivery article for placement over or into an orifice, cavity or surgically created space that comprises a first layer having a first surface which is contactable with the wound, cavity, or orifice and has disposed thereon a sulfonated styrene polymer. The first layer may be impregnated with or coated with the sulfonated styrene polymer. A second layer of the wound dressing or drug delivery article may be a solid film, a perforated film, a fiber or strand of natural or manmade material, a woven fabric or gauze of natural or manmade material, a nonwoven fabric of natural or manmade material, and/or a knitted fabric of natural or man made material. The invention also relates to a method of treating a wound comprising applying to a wound in need of treatment, a wound dressing or packing, the wound dressing or packing comprising a layer having a first surface contactable with the wound and having a sulfonated styrene polymer disposed thereon. In yet another aspect, the invention relates to a method of treating a medical condition using a vaginal or rectal insert, where the insert is comprising a layer having a surface contactable with the wound and having a sulfonated styrene polymer disposed thereon. In one embodiment, a wound dressing, wound packing or cavity insert of the present invention additionally comprises a therapeutic agent. Preferred dressing embodiments include individual square or rectangular sheets, patches, films, rolled sheet, fiber, strand, or rope forms. Preferred therapeutic agents are antibiotics such as gentamicin, antibacterial agents such as quaternary ammonium ions, silver sulfadiazine, or polystyrene sodium sulfonate, anesthetics such as lidocaine; inhibitors of protease function such as doxycycline, other tetracyclines, secretory leukocyte protease inhibitor (SLPI), or Aprotinin (a 57 amino acid serine protease inhibitor); growth factors such as platelet-derived growth factor, spermicides such as nonoxynol-9; antiviral agents such as polystyrene sulfonate, dextran sulfate or other polyanions, Vidarabine or acyclovir; antifungal agents such as Clotrimazole or Miconazole; antiparasitic agents such as Ivermectin; steroidal and non-steroidal anti-inflammatory agents such as dexamethasone and Ketoprofen; anti-histamines such as fexofenadine or benadryl, analgesic agents such as NSAIDs naproxen or acetaminophen, antineoplastic or antiproliferative agents such as sirolimus or paclitaxel, hormones such as estradiol; kerolytic agents such as salicylic acid o lactic acid, tranquilizers, vitamins such as vitamins E or A; base-pair nucleotides, genes, DNA, RNA and/or cytokines. In another embodiment a drug delivery insert for placement into an orifice, cavity or surgically created space comprises a therapeutic agent for delivery into the surrounding tissue. A preferred embodiment for a vaginal insert is that of a tampon having a sulfonated styrene polymer disposed thereon and further compounded with a therapeutic agent such as miconazole for the treatment of candida or metronidazole for the treatment of bacterial vaginosis. A preferred embodiment for the treatment of periodontal disease is a fiber or strand coated with a sulfonated styrene polymer and loaded with doxycycline. A preferred embodiment for a drug delivery patch for placement on or near a tumor, includes 5-fluorouracil (5FU) loaded into a sulfonated styrene polymer film or fabric coated with a sulfonated styrene polymer. In yet another embodiment a medical article/device for placement into a surgically created space, or existing space within the body, requiring surgery to access. A preferred embodiment includes a stent for opening a vein, artery, or mucosal surface such as in the GI tract. Additionally, the stent having a styrene copolymer hydrogel disposed thereon and further compounded with a therapeutic agent for minimizing the proliferation of tissue. Preferred agents include paclitaxel and sirolimus or other appropriate immunosuppressive agent. In yet another aspect, the present invention relates to a method of manufacturing an implantable medical device, the method comprising coating at least one surface of the implantable medical device with a sulfonated styrene polymer containing at least one of sirolimus, paclitaxel, or other antineoplastic or immunosuppressive agent. Coating methods include dipping, conventional spraying, painting, or electrospraying or electroprocessing.

The delivery of certain pharmaceutical agents can be readily accomplished from the mucosal surfaces inside of the rectum, GI track, cheek (buccal), and vagina. In another embodiment a drug delivery insert for placement into an orifice or cavity having a mucosal surface such as the inside of the cheek (buccal) with a wafer or film, or into the GI tract as with a tablet, or into the vagina or rectum with a suppository delivery vehicle is envisioned. Furthermore, there are several disease states that require the delivery of pharmaceutical agents directly into the vagina. The use of creams and suppositories can in some instances be messy, inefficient, and in some cases culturally unacceptable. The use of a controlled-release tampon is proposed for the treatment of some diseases of the vaginal tract and as a post intercourse birth control device.

Controlled Release: The literature has stated that the release of protease inhibitors into the chronic wound may be beneficial in restoring the proteinase/antiproteinase balance needed to avoid degradation of growth factors and effectively accelerate healing of chronic wounds (Herouy, et al. European J. Dermat., 10 (3), April-May 2000, 173-80). The active agents necessary to inhibit the action of wound proteinases are applied to the wound site directly and in controlled fashion from a sulfonated styrene polymer wound dressing. The invention includes methods of linking a protease inhibitor, such as doxycycline, to the hydrogel wound dressing through an ion-exchange interaction between the sulfonate group of the sulfonated styrene polymer and the drug of interest or with a biomolecule protease inhibitors such as aprotinin or SLPI, or with a growth factor such as platelet derived growth factor via the hydration of the dehydrated sulfonated styrene polymer dressing in an aqueous solution of the biomolecules(s) of interest.

Additionally, the polyanionic sulfonated styrene polymer is an intrinsic sequesterant for divalent cations such as Zn²⁺ found in the catalytic domains of endopeptidases such as neutrophil collagenase (MMP-8). The competitive binding of zinc by the numerous and organized sulfonate groups of the sulfonated styrene polymer, i.e. a sequesterant, is expected to disrupt the catalytic domain of the endopeptidase thus rendering the enzyme inactive. Furthermore, the polyanionic nature of the sulfonated styrene polymer and the prevalence of the negatively charged sulfonate groups along the backbone of the polymer enables the dressing to attract, bind, and deactivate electropositive species such as neutrophil elastase, a detrimental wound proteinase. Furthermore, the anionic structure provides a stabilizing environment for incorporated biomolecules such as proteins and peptides.

Therefore, it is one object of this invention to provide methods and compositions for the enhanced treatment of mammalian wounds comprising the application of protease inhibitors and sequesterants from the sulfonated styrene polymer dressing.

The present invention takes advantage of the unique chemical structure, processing, and mechanical property advantages of sulfonated styrene polymers. In particular, the ability to coat man-made and natural substrates with sulfonated styrene-containing copolymers, and bind cooperatively bind molecular (therapeutic) species with certain ionizable functionalities as precursors to the fabrication of drug delivery and healing articles.

The wound dressing component of the present invention is based upon the published scientific belief that inhibitors and sequestrants of proteases may be used as healing accelerants of chronic wounds. These may be physically applied on wound dressings, or in the alternative may be ionically or covalently conjugated to a wound dressing material for purposes of sustained release of active agent or sequestration of endogenous constituents from the wound environment. The term protease inhibitor is meant to include those materials that affect a diminution in protease activity in the wound environment. This technology is broadly applicable to all forms of chronic wounds including diabetic ulcers, venous ulcers, and decubitus bedsores.

The dose of inhibitor or sequestrant required on the wound dressing to promote accelerated healing in the patient ranges from about 0.025 mg/gram of dressing material to about 250 mg/gram of dressing material per day. For example, a continual dosage of doxycycline to maintain the concentration at the same concentration known to be effective (serum) following oral dosing, ca. 30 μM, is desirable. Other factors that are crucial in healing include patient health, wound type, and specific protease inhibitor/sequestrant utilized. The amount of active agent required can be readily determined by those skilled in the art.

The term patient used herein is taken to mean mammals such as sheep, horses, cattle, pigs, dogs, cats, rats, mice and primates, including humans.

The vaginal drug delivery component of the present invention is based upon the belief that a tampon provides the easiest method for delivering a drug into the vagina. Tampons are commonly used in the western world for menstrual fluid management, are easily placed, handled and easily removed for disposal. A natural fiber tampon coated with a composition of a sulfonated styrene-containing copolymer and an appropriate therapeutic agent and further hydrated with an aqueous solution yields a soft, supple, and comfortable drug delivering vaginal insert.

Sulfonated styrene polymers are strong enough acids to protonate amino acids such as proline, arginine and others. For serious wounds such as pressure sores, diabetic ulcers, and venous ulcers, the stimulation of angiogenesis is desirable due to the ischemic nature of many of these wounds. Arginine is a good choice for a therapeutic agent because it shows multiple and potent biological activities. Beneficial effects on wound healing and immune system have been reported, making arginine a potential therapeutic agent. It is also a secretagogue acting on pituitary, pancreas, and even adrenal function. These activities give rise to molecules such as nitric oxide and perhaps glutamate derived from it. Nitric oxide modulates immune function and lymphocyte activities in wounded tissues. The fibroblast-collagen synthesis required for healing is activated by cytokines release. A direct action is exerted by arginine on pancreatic B cells for insulin release. Arginine stimulates pituitary secretion of GH and LH by acting at a suprapituitary level through a somatostatinergic tone decrease and through an increase of LHRH production. The implication of nitric oxide in LHRH stimulation has been demonstrated. It could also to explain the somatostatinergic tone decrease.

Angiogenesis is a complex process that involves the activation of quiescent endothelial cells (ECs) to a proliferative and migratory phenotype and, subsequently, their redifferentiation to form vascular tubes. We hypothesized that NO contributes to angiogenesis by terminating the proliferative action of angiogenic growth factors and initiating a genetic program of EC differentiation. Human umbilical vein ECs (HUVECs) and calf pulmonary artery ECs (CPAECs) were grown directly on plastic dishes or on three-dimensional fibrin matrices. In the absence of fibrin, treatment with NO-donor compounds, such as S-nitroso-N-acetylpenicillamine (SNAP, 0.1 and 0.4 mmol/L), produced a dose-dependent inhibition of proliferation in both cell lines, whereas the inhibition of endogenous NO production using N(G)-nitro-L-arginine methyl ester (L-NAME, 1 mmol/L) or N(G)-monomethyl-L- arginine (L-NMMA, 1 mmol/L) significantly increased proliferation of the CPAECs. The addition of basic fibroblast growth factor (bFGF, 30 ng/nL) increased the expression of endothelial NO synthase mRNA and the production of NO in both cell types when cultured on three-dimensional fibrin gels and produced profound morphological changes characterized by the appearance of extensive capillary-like vascular structures and the loss of EC monolayers. These changes were quantified by measuring total tube length per low-power field (X 100), and a differentiation index was derived using the ratio of tube length over area covered by residual EC monolayer. In the absence of additional angiogenic factors, the differentiation index was low for both HUVECs and CPAECs (control, 1.16plus or minus0.19 and 2.07plus or minus0.87, respectively). Treatment with bFGF increased the differentiation index significantly in both cell types (10.59plus or minus2.03 and 20.02plus or minus5.01 for HUVECs and CPAECs, respectively; P<0.05 versus control), and the addition of SNAP (0.4 mmol/L) mimicked the angiogenic response to bFGF (8.57plus or minus1.34 and 12.20plus or minus3.49 for HUVECs and CPAECs, respectively; P<0.05 versus control). Moreover, L-NAME inhibited EC tube formation in response to bFGF in a dose-response manner, consistent with a role of endogenous NO production in EC differentiation in this angiogenic model. These findings suggest that NO may act as a crucial signal in the angiogenic response to bFGF, terminating the proliferative actions of angiogenic growth factors and promoting EC differentiation into vascular tubes.

Proline readily forms “salts” with the acid form of the SSEBS polymer. This polymer has been shown to provide some advantages in tempering the acidity of the polymer while providing a soluble salt form for improved processing, particularly useful for formulations that may include a therapeutic agent that may be susceptible to acid. For example, the acid labile molecule paclitaxel was released intact, from a proline derivative of 29 mole % sulfonated SEBS, whereas when the acid form of the polymer was utilized the drug was hydrolyzed and degraded Arginine and other amino acid derivatives of up to 60% SSEBS have also been prepared, and incorporation of the amino acid was confirmed via FTIR spectroscopy.

In spite of recent advances in our understanding of the basic mechanisms of wound healing, knowledge of the factors leading to chronic ulcers remains limited. In the last decade molecular biological investigations performed in these ulcers focused on proteolytic properties of proteases and their significance in the remodeling process of chronic wounds. Among distinct populations of enzymes, it is well recognized that matrix metalloproteinases play an outstanding role due to their capability to degrade essential structural proteins constituting the architecture of human skin. Different investigations provided evidence that matrix metalloproteinases participate at different stages of the ulcerative process, from their formation with the initial epithelial defect until ulcer resolution and repair. Therefore we may provide insight into general tissue alterations caused by matrix turnover, into the family of matrix metalloproteinases and their activation as well as inhibition.

Matrix metalloproteinases (MMPs) play an important role in the remodeling of the extracellular matrix. Recent studies have increased the list of biological processes in which matrix metalloproteinase appear to be involved, and in several cases pointed to processes that do directly involve matrix remodeling. These enzymes constitute a family of several zinc-dependent endopeptidases which are expressed at low levels in normal adult tissues. They are upregulated during different normal and pathological remodeling processes such as embryonic development, tissue repair, inflammation, tumor invasion and metastasis. Matrix metalloproteinases are known to be proteases that can cleave collagen macromolecules, which are of significant importance in maintaining the architecture and integrity of skin.

Matrix metalloproteinases belong to a growing family of soluble and membrane-bound endopeptidases which degrade important structural proteins. The catalytic domain, which contains the active Zn²⁺ and stabilizing Ca²⁺-binding site, is required for proteolytic activity and for membrane binding [12]. Proteolytic properties of these enzymes are controlled by transcriptionally regulated protein synthesis as well as by post-translational modification of the synthesized proteins. Most matrix metalloproteinases are constitutively expressed in vitro at low levels by different cell types, such as keratinocytes, fibroblasts, macrophages, endothelial cells, mast cells, eosinophils and neutrophils. Matrix metalloproteinases are induced at transcriptional level by a variety of mediators such as interleukin-1and -6 (IL-1 and IL-6), tumor necrosis factor-alpha (TNF-alpha), epidermal growth factor (EGF), platelet derived growth factor (PDGF), fibroblast growth factor (FGF), and transforming growth factor-beta (TGF-beta). At present the matrix metalloproteinase family consists of several structurally related members each of which can be categorized according to the primary structure and substrate specificity into distinct subgroups of collagenases, gelatinases, stromelysins and membrane type matrix metalloproteinase (MT-MMP). Matrix metalloproteinases display major domain structures. Each matrix metalloproteinase subtype consists of a propeptide, a catalytic domain containing a Zn²⁺-binding site, and a hinge region connected to four pexin like domains. Collagenases currently consist of the interstitial collagenase (MMP-1), the neutrophil collagenase (MMP-8) and collagenase-3 (MMP-13). These interstitial collagenases are capable of degrading native fibrillar type I, II, III and V collagen macromolecules. The interstitial collagenase-1 (MMP-1) degrades type III collagen whereas MMP-8 is more effective in degrading type I collagen. Collagenase-3 (MMP-13) is able to degrade type II collagen six-fold more effectively than type I and III collagens. Collagenase-3 (MMP-13) displays stronger gelatinolytic activity than MMP-1 and MMP-8 and is capable of degrading type IV, IX, X and XIV collagens, tenascin C and fibronectin.

The antimicrobial activity of silver ion is well defined. Silver ion rapidly kills microbes by blocking the cell respiration pathway. The speed of action is almost instantaneous once the silver reaches the microbe. The efficacy of microbe killing is based not only on the amount of silver ion present, but thought to be due to the presence of other silver radicals generated by a silver releasing product.

Because of mechanism of action, microbial resistance to silver itself has not been reported. In addition, silver has repeatedly been shown to be non-toxic to human cells. Toxicity occurs from the complexes used to deliver silver such as nitrate and sulfadiazine.

The anti-inflammatory effects of silver ion on a wound have been recognized for centuries. Most of the reports are purely descriptive in nature identifying the decrease in erythema and increased healing. A number of biochemical effects related to the effects of silver on wound healing have been documented over a decade ago. However, only recently with the new concepts on wound healing and healing impairment, can a mechanism of action be presented. One of the latest major foci of wound healing has been the relationship between tissue destruction by a group of collagen destroying enzymes known as MMP and tissue repair which is stimulated in part by growth factors. An excess of MMP activity has been reported in burn wounds and in chronic wounds.

Action of the MMP's is dependent on the availability of free Zinc, as free zinc activates the proenzyme form of the protease. Silver is believed to decrease surface zinc (by dilution), which may decrease excess MMP activity and hence (potentially) increase healing rate. Recent reports suggest that silver (as delivered by the pure silver system ACTICOAT wound dressing) decreases MMP activity. Additionally, silver purportedly increases wound surface calcium, which should stimulate epithelialization. Furthermore, as silver ion is dumped into the wound, at the surface of the dressing there are fewer inflammatory components (MMPs) and a decrease in inflammation. For this reason silver dressings are thought to be good for the treatment of burns because decreasing excessive metalloproteinase (MMP) activity, as found in severe burns, is possibly due to decreasing available zinc ion. Sulfonated styrene copolymer hydrogels are ion exchange materials and are good complexers of divalent cations such as Ca⁺², Mg²⁺, Mn²⁺, Zn²⁺ as well as others. These polymers may disrupt the enzyme active site by complexing Zn²⁺ and leading to a novel mechanism of inhibition. The polymers may be used to fabricate dressings that down-regulate out-of-control MMP function inhibiting enzyme function. Removal of one or the other of the above ions represents one way of inhibiting the enzyme function. In addition to the chelation (by Aegis sulfonated biomaterials) approach to enzyme regulation, hydrogel dressings based on sulfonated styrene polymers may employ the use of a therapeutic proteinase inhibitor such as doxycycline. Doxycycline is a tetracycline antibiotic that is a known broad spectrum, non-specific inhibitor of matrix metalloproteinases. Furthermore, doxycycline binds nicely to both the acid and salt forms of sulfonated styrenic copolymers. The doxycycline analogs have provided continual delivery of the therapeutic for more than 48 hours for a polyester supported dressing coated at a level of 102 mg/in² and loaded with doxycycline.

The doxycycline analog 102-doxhas been shown to be very effective against MMP-8, collagenase. In another trial, collagenase was been completely neutralized, whereas Promogran, a Johnson & Johnson product purported to lower collagenase, has yielded approximately 70% inhibition under these conditions. It is very plausible that given Promogran's construction, a composite of collagen and ORC, this dressing doesn't inhibit collagenase or MMPs in general. And that what is shown in this experiment is the inability of the enzyme to digest all of the available (soluble) collagen substrate in the dressing.

Strong (as well as weak) cation exchange resins, such as Dowex® or Amberlite®, and resins such as ProPac® may also be employed in wound dressings aimed at controlling MMPs. One such method would be to take the resin and in dry form crush it to powder. Next the powder may be added to a standard hydrogel formulation such as a hydrogel requiring crosslinking via gamma radiation or free-radical (catalyst) initiation. Additionally, the cation exchange resin powder may be added to a polyurethane hydrogel for coating of fabric, or addition to hydrocolloid, alginate, std. polymerizable Hydrogel, Aegis sulfonated copolymers as in patents U.S. Pat. No. 5,840,387, 11-24-98 and U.S. Pat. No. 6,306,419, 11-03-01), or combined into a composite dressing. There are numerous other methods and/or formulations that may be utilized for placing the strong cation exchange resin directly in contact with a wound or wound fluid beyond the few examples mentioned herein and will be apparent to those skilled in the art.

The polyanionic nature of polysulfonated styrene copolymers not only provides a biocompatible and stabilizing environment for biomolecules, such as proteins, peptides and the like that are of interest for delivery to the patient, we have shown that sulfonated SEBS (60%) sulfonation is a good inhibitor of neutrophil elastase, a serine protease prevalent in the chronic wound environment. When exposed to 30 milliunits of neutrophil elastase, several formulations of the sulfonated copolymer styrene-ethylene-butylene-styrene (SSEBS) inhibited the enzyme by as much as 40% as seen for the ammonium salt (SSEBS-NH₄). Inhibition by approximately 33% was observed for the SSEBS-Na⁺ analog.

### EXAMPLES

### Example 1A

SSEBS coated polyester fabric. Preparation of SSEBS sodium and SSEBS ammonium salts: A woven PET fabric (6″.times.6″) was dipped in a 5% solution in THF of a styrene-ethylene-butylene-styrene triblock copolymer (SEBS) sulfonated to 65% mole percent, based on styrene, removed and allowed to dry on a sheet of PTFE. This dip coating process was repeated twice. (Higher solids concentrations can be utilized and require fewer dips overall.) The dried, coated fabric was placed into an aqueous solution of sodium bicarbonate (NaHCO.sub.3) for about 1 hour to yield the sodium salt of the sulfonated styrene polymer, SEBS sodium sulfonate.

### Example 1B

SSEBS coated polyester fabric. Preparation of SSEBS ammonium salt: A woven PET fabric (6″.times.6″) was dipped in a 5% solution in THF of a styrene-ethylene-butylene-styrene triblock copolymer (SEBS) sulfonated to 65% mole percent, based on styrene, removed and allowed to dry on a sheet of PTFE. This dip coating process was repeated twice. (Higher solids concentrations can be utilized and require fewer dips overall.) The dried, coated fabric was placed into a solution of aqueous ammonia (NH₄OH) for about 1 hour to yield the ammonium salt of the sulfonated styrene polymer upon drying. The ammonium salt is desirable because ammonia is volatile and evaporates upon drying.

### Example 2

SSEBS and Benzyltrimethylammoniumchloride: The sodium salt of SSEBS, supported on PET fabric, as prepared in example 1 above, was placed in an aqueous solution of benzyltrimethyl ammonium chloride. The composite was allowed to hydrate and equilibrate, yielding the benzyltrimethyl ammonium (BTMA) salt of SSEBS (SSEBS-BTMA). BTMA acts as a preservative for the dressing, in addition to providing disinfecting and antiviral properties. The inclusion of other alkyl ammonium salts is straightforward based on this example.

### Example 3

SSEBS And Polystyrene Sodium Sulfonate: SSEBS (alternatively CaCl₂ can be added to bind the polystyrene sulfonate to the SSEBS via ionic interaction).

### Example 4

SSEBS and Nonoxynol-9 (Antiviral/spermicide): Nonoxynol-9 is added to a SSEBS lacquer. The structure of the therapeutic agent lends itself well to solubility in the SSEBS backbone, providing an excellent means for diffusion-controlled release of this agent.

### Example 5

SSEBS Coated Tampon And Miconazole: Miconazole is an amidine antifungal agent. The amidine moiety lends itself well to protonation by SSEBS to yield a salt. The salt form will slow diffusion of miconazole resulting in longer term (controlled) delivery. A Tampax tampon is coated with a 10% solids solution of 60% SSEBS and the tampon is allowed to dry. The tampon is placed in a normal saline solution (100 mL) and allowed to hydrate. To the solution, 100 mL of ammonium hydroxide is added and the container covered. The tampon was allowed to soak at room temperature for 3 hour. The tampon was removed and placed onto a PTFE sheet and allowed to air dry. Miconazole nitrate (0.1 g/cc) was prepared and the tampon allowed to hydrate for 24 hours at room temperature. The tampon was removed and allowed to air dry.

### Example 6

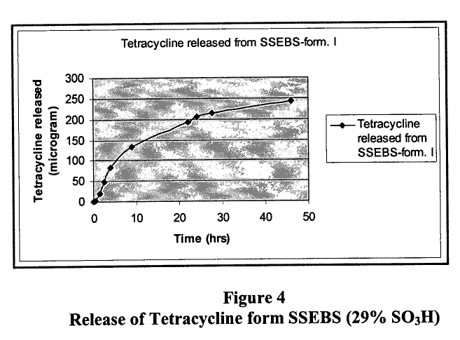

SSEBS And Doxycycline/Tetracycline: A SSEBS coated PET fabric was prepared as described in Example 1A, and the ammonium salt prepared as described in 1B. A solution of tetracycline hydrochloride (0.1 g/cc) was prepared and the fabric allowed to hydrate for 24 hours at room temperature. The sample was dried at room temperature. UV absorption data of a 0.1 g sample yielded the drug release profile detailed in FIG. 4.

### Example 7

SSEBS and silver. A SSEBS coated PET fabric was prepared as described in Example 1A, and the sodium salt prepared as described in 1A. A solution of Silver nitrate (0.1 g/cc) was prepared and the fabric was allowed to hydrate in it for 24 hours at room temperature in an aluminum foil protected container. The sample was removed and rinsed in DI water several times and allowed to soak in DI water for 24 hours (2×). The material was removed and dried at room temperature. Aegis sulfonated SEBS (SSEBS) polymer was coated onto a woven PET substrate at a loading of 102 mg/in². The dried fabric was placed into a solution of NaHCO₃ (0.5M) and warmed to 40° C. Deprotonation of the polymer is evident from the evolution of CO₂ at the surface of the dressing. The dressing is removed when CO₂ evolution ceased, rinsed in DI water and placed into a solution of AgNO₃ (0.2M) for 24 hours and removed and washed in DI water. A dry piece of SSEBS film that had been prepared using the same conditions was sent for elemental analysis and silver was determined to be 9.56% by weight. The theoretical value for 100% incorporation is ca. 19%. Thus, there is significant room for greater incorporation and may likely be accomplished by using a stronger base such as NaOH or NH₄OH.

Microbial Challenge: Six 1.0 mm discs were fashioned from each test dressing. ATCC strains of Pseudomonas aeruginosa (ATCC #27853); Staphylococcus aureus (ATCC #29213) Enterococcus faecalis and Escherichia coli were standardized to a 0.5 MacFarland standard and inoculated to a Mueller-Hinton agar plate. The doxycycline and Ag⁺ discs were placed onto the inoculated plate along with an unimpregnated control disc and incubated at 37° C. for 24 hours. Zones of inhibition were measured in mm. For quantitative assessment, 4 tubes (TS broth) for each organism were each inoculated with 100 μL of a 0.5 MacFarland Standard. From 1 to 3 discs were introduced into each one of the 3 tubes with the 4^{th} tube utilized as a positive control. All tubes were incubated at 37° C. for 72 hours. At each 24 hr interval an aliquot (0.01 uL calibrated loop) was streaked out on appropriate media to determine the quantitative count for each test product.

Results: Table 1 provides the data for the modified Kirby-Bauer disc method. The quantitative assessment was more promising even at 1 disc. Both visually and quantitatively all tubes with both Ps. aeruginosa and S. aureus had no growth at the end of 48 hours. However at 72 hours, counts for Ps. Aeruginosa, S. aureus, Ec. Faecalis and E. coli were TNTC as were the controls.

Conclusion: While the modified Kirby Bauer disc method showed inhibition of both organisms by the doxycycline:H⁺ and Ag⁺ discs, the quantitative assessment showed that 1 disc was comparable to 2 or 3 disc's, a clear indication that diffusion of the anti-infectives through the solid media may be impeded as a consequence of solubility of the anti-infective in the agar. Each product in a liquid environment was exceptionally effective for at least 48 hours as were the Silverlon and Acticoat dressing samples. In order to improve these data, higher drug and metal ion loadings may be formulated.

TABLE 1

| Organism | Doxycycline Zone | Ag⁺ Zone | Control Zone |
|---|---|---|---|
| Ps. Aeruginosa | 14 mm | 10 mm | 0 mm |
| S. aureus | 28 mm | 9 mm | 0 mm |
| Ec. Faecalis | 18 mm | 0 mm | 0 mm |
| E. coli | 22 mm | 9 mm | 0 mm |

### Example 8

Controlled Release: Tetracycline was ion exchanged into a 10 mg sample of sulfonated SEBS (SSEBS-sodium sulfonate, 29% sulfonation) and the SSEBS film released 250 μg (2.5% by weight) of the drug over a 48-hour period into Tris buffer. The release profile was devoid of the characteristic “burst-release” effect observed for diffusion-controlled devices. Thus, any charged tetracycline derivatives may have a similar release profile.

The sulfonated elastomers are derived from the group of polymers including styrene butadiene, styrene-ethylene-butylene-styrene (SEBS), styrene-ethylene (SE), styrene-isoprene, styrene-isoprene-styrene (SIS), styrene-isobutylene-styrene (SIBS), styrene-ethylene-propylene-styrene (SEPS). The styrene alkene random copolymers such as styrene-ethylene can also be formulated to include higher alkenes such as propene, butene, pentene, hexene, heptene etc. with the limit being octadecene.

Furthermore, blends of the above mentioned materials can be formulated to alter properties or adjust sulfonation levels. For example, sulfonated styrene-ethylene-butylene-styrene (SSEBS) may be formulated in the following fashion as detailed in Example 9.

### Example 9

For two sulfonated polymers, of different sulfonation levels of 60% and 20% and preferably prepared from the same lot of starting SEBS although not necessary, the blending of these two materials may be carried out in order to provide a material with a final sulfonation level less than 60% but greater than 20%. The combination of equal parts of the above carried out by combining two separate lacquers or by dissolution of the combined solids (60% and 20% sulfonated) would yield a final blend with an average sulfonation level of 40%. Adjusting the weighted average accordingly allows variation of the final blend as expected. For example, the combination of 80 g of 20% SSEBS with 20 g of 60% SSEBS yields a final material/blend with a sulfonation level of (0.8×20)+(0.2×60)=28%.

Example 10 details how composite materials including sulfonated SEBS or SE are prepared and what their advantages are.

### Example 10

Blending of a sulfonated polymer, such as SSEBS or SSE with a non-sulfonated starting material such as SEBS, SE, or other polymer such as polyurethane is straightforward. In this example the resultant material is phase separated. When the non-sulfonated polymer is present at a high enough loading to result in a continuous phase, dramatically improved mechanical properties are imparted to the blend.

Example 11 provides a detailed description of the preparation of conformally coated medical devices with sulfonated coatings of high uniformity.

### Example 11

A stent is coated with parylene or poly(benzcyclobutene). Chemical vapor deposition yields coatings that are highly controllable and uniform. The thickness can be controlled by the time in the deposition chamber and other variables of the coating process. The coated stent (with the intractable polymer coating) is then placed into a solvent such as dichloroethane or stable fluorinated solvent such as Ausimont's Galden or Fomblin perfluorinated polyether, and acetyl sulfonate is added. The sulfonation reaction is allowed to proceed for the appropriate time, the stent is removed, washed in isopropanol and rinsed with water in order to remove any residual solvents. The duration of exposure provides control of the sulfonation level. This procedure is easily adapted to treat any similarly coated device such as a shunt, can, heart valve leaflet, introducer, guidewire, surgical tool/instrument etc.

### Example 12

A stent or other medical device such as a shunt is coated by spraying, dipping, or painting from an appropriate solvent with SEBS, SIBS, SE or other aromatic (benzenoid) ring containing polymer. The device is placed into a nonsolvent for the polymer such as Ausimont's Galden or Fomblin perfluorinated polyether (i.e. a nonsolvent for the polymer, and acetyl sulfate is added. The sulfonation reaction is allowed to proceed for the appropriate time, the device is removed, washed in isopropanol and rinsed with water in order to remove any residual solvents. The duration of exposure provides control of the sulfonation level. This procedure is easily adapted to treat any similarly coated device such as a shunt, can, heart valve leaflet, introducer, guidewire, surgical tool/instrument etc.

### Example 13

The polyester sewing cuff of a pyrolytic carbon heart valve was coated with 60% sulfonated SEBS (5% solids×3 dips). The fabric was allowed to air dry for 48 hours at which time the entire valve was submerged into saturated aqueous NaHCO₃ and allowed to sit overnight. The valve was removed rinsed with copious amounts of water and allowed to sit in DI water overnight. The valve was transferred to a solution of AgNO₃ (0.5 g/mL) in a beaker wrapped in aluminum foil in order to prevent light from entering. The valve was allowed to soak overnight in the absence of light. The valve was removed from the AgNO₃ solution and placed directly into a solution of sodium bisulfite (NaHCO₃) heated to 70° C. Immediately, white & gray colors begin to appear. After 15 minutes the sewing ring has taken on a deep gray color indicating the presence of metallic silver. The ring remains soft and supple when hydrated. SEM analysis of the fabric reveals that the silver has permeated the material through and through and that the particles in the material are undetectable by SEM.

### Example 14

Preparation of SSEBS-amino acid Ionologs: An aqueous (sterile DI) solution of the amino acid is prepared and the SSEBS polymer is added directly and stirred for 24 hours. The polymer is removed, rinsed/soaked in sterile DI water and air-dried for at lease 24 hours. Following drying, the amino acid derivative may be dissolved in THF, CHCl₃, or combinations thereof.

An organic solution of SSEBS polymer is prepared in THF or solvent combination and the lacquer is stirred with an excess of amino acid for 24 hours. At this point, the amino acid is filtered from the lacquer and the SSEBS isolated in order to prove incorporation has occurred.

Observation: A discriminate amount of SSEBS lacquer (ca. 5 CC, 29% sulfonated in THF/CHCl₃, 10% solids) was combined with 5 CC of toluene without any precipitation of the polymer. Thus high solids solutions could be cut with toluene in order to provide coating-drug combination options.

Solvent Switching: The solution should be placed onto a rotary evaporator, water bath ca. 50-60° C. and THF and CHCl₃ preferentially removed while observing to see if the polymer precipitates. The rationale here is that once the polymer has dissolved into a polar solvent (THF), the H-bonding between SO₃H groups has been disrupted and the EB block may dominate the solubility dynamics and allow the inclusion, and prevalence of a non-polar solvent. With more highly sulfonated materials, swell in THF, add chloroform and n-propanol and heat slightly, and remove THF/CHCl₃ via rotary evaporation to the appropriate solids concentration.

1. A method for inhibiting elastase and/or collagenase in a wound, said method comprising contacting the wound with a composition comprising a combination of a sulfonated styrene copolymer and a tetracycline.

2. A method according to claim 1, wherein the tetracycline is doxycycline.

3. A method for inhibiting elastase in a wound, said method comprising contacting the wound with a composition comprising a sulfonated styrene copolymer in salt form.

4. A method according to claim 3, wherein said composition additionally comprises a tetracycline.

5. A method according to claim 1, wherein the composition is disposed on a surface of a wound dressing.

6. A method according to claim 5, wherein the wound dressing comprises a substrate selected from a foam, a woven fabric, a knit fabric, and a nonwoven fabric.

7. A composition comprising a combination of a sulfonated styrene copolymer and a tetracycline.

8. A composition according to claim 7, wherein the tetracycline is doxycycline.

9. A composition according to claim 7, wherein at least a portion of the sulfonated styrene copolymer is in the form of a salt.

10. A composition according to claim 7, wherein at least a portion of the sulfonated styrene copolymer is in the form of an ammonium salt.

11. A composition comprising a combination of a sulfonated styrene copolymer and an amino acid.

12. A composition according to claim 11, wherein the amino acid is proline.

13. A composition according to claim 11, wherein the amino acid is arginine.

14. A process for manufacturing articles comprising of at least one sulfonated styrene copolymer, said article selected from tubes, sheets and 3-D constructs, said process comprising electrodepositing the sulfonated styrene polymer to form the article.

15. A method for controlling biological organisms on a porous surface, said method comprising forming a coating, comprising a salt of a sulfonated styrene copolymer, on the porous surface.

16. A method according to claim 15, wherein forming a coating comprises coating the porous surface with the sulfonated styrene polymer in acid form and converting the acid form of the sulfonated styrene copolymer to the salt form.

17. A method according to claim 15, wherein the sulfonated styrene polymer is an ammonium salt.

18. A method according to claim 1, wherein the porous surface comprises fabric or paper.

19. A method according to claim 1, wherein the porous surface comprises an article selected from a garment, an air filter, a gas filter, a laboratory work surface, or laboratory wipe.

20. A composition according to claim 1, wherein the styrene sulfonate copolymer comprises residues derived from an olefin comonomer.

21. A composition according to claim 1, wherein the olefin comonomer is selected from ethylene, butylene, isobutylene, butadiene, isoprene and combination thereof.

22. A composition according to claim 21, wherein the sulfonated styrene copolymer is hydrogenated to reduce unsaturated olefin residues

23. A composition according to claim 1, wherein the sulfonated styrene copolymer is a block copolymer.

24. A composition according to claim 1, wherein the sulfonated styrene copolymer is a sulfonated styrene-ethylene-butylene-styrene triblock copolymer.
